(19) United States
(12) Patent Application Publication
(10) Pub. No.: US 2003/0207304 A1
(43) Pub. Date: Nov. 6, 2003

(54) Glycerol-doped aerogel coatings as biological capture media

(76) Inventors: Eric Black, Charlottesville, VA (US); Kathy Terlesky, Charlottesville, VA (US); Christopher Earle, Ruckersville, VA (US)

Correspondence Address: John G. Posa, Gifford, Krass, Groh, Sprinkle, Anderson & Citkowski, P.C., 280 N. Old Woodward Ave., Suite 400, Birmingham, MI, 48009-5394, US

(21) Appl. No.: 10/402,047

(22) Filed: Mar. 28, 2003

Related U.S. Application Data
(60) Provisional application No. 60/375,790, filed on Apr. 26, 2002.
(60) Provisional application No. 60/376,905, filed on May 1, 2002.

(51) Int. Cl. C12Q 1/68; C12M 1/34; B05D 3/00

(52) U.S. Cl. 435/6; 435/287.2; 427/2.11

(57) ABSTRACT

The present invention provides a polyol-doped aerogel material with improved properties for trapping biological particles and facilitating analysis and identification thereof. The polyol-doped aerogel material can be used in combination with a substrate by coating or otherwise depositing the material thereon. The present invention provides a method for detecting a biological particle involving use of a polyol-doped aerogel material.

### REFERENCE TO RELATED APPLICATIONS

This application claims priority from U.S. Provisional Patent Application Serial Nos. 60/375,790, filed Apr. 26, 2002, and 60/376,905, filed May 1, 2002, the entire content of both of which are incorporated herein by reference.

### FIELD OF THE INVENTION

This invention relates to materials and methods for collection of biological particles using polyol-doped aerogels as capture media. In particular the invention relates to a glycerol-doped aerogel materials and methods of use thereof as capture media.

### BACKGROUND OF THE INVENTION

Existing methods of collection for air-borne bacteria include impaction devices (Burkhard spore trap, Andersen sampler, Rotorod), impingement devices (AGI-30), and filtration devices. Impaction devices collect air-borne material either on a bacterial culture medium (such as an agar plate), on a silicone surface, or on a filter.

Older methods are prone to complications in the analysis of collected biological material, whether bacteria, spores, or fungi. This is due to an inability to remove the trapping media which interferes with the processing necessary to complete identification and quantification assays.

Although silicone is an effective collection material, it is incompatible with removal and molecular analysis of collected biological particles. Glycerol is a known biocompatible solute suitable for bacterial collection; however, it is viscous, and not compatible with forced-air collection associated with filtration or impingement devices. Thus, there is a continuing need for a capture medium for biological particles which is compatible with particle viability and detection assays.

### SUMMARY OF THE INVENTION

An improved biological particle capture medium is provided which includes a polyol-doped aerogel deposited on a substrate. The inventive polyol-doped aerogel includes an oligomeric silicate having the formula:

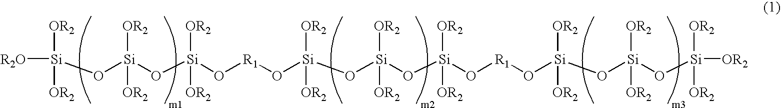

wherein R1 and R2 are each independently a C₁₋₂₀ alkyl, aryl, arylalkyl, substituted aryl, alkenyl, substituted alkenyl, alkynyl, substituted alkynyl, cycloalkyl or heterocycle group and wherein R2 may be hydrogen.

Optionally, R1 and/or R2 further include a pendant moiety selected from the group consisting of: hydroxyl group, C₁-C₆ linear alkyl group, alkoxy group, C₃₋₆ branched alkyl group, cyclopentyl group, and cyclohexyl group

The invention further provides an improved biological capture medium wherein the polyol-doped aerogel includes a glyceryl-bridged oligomeric silicate having the formula:

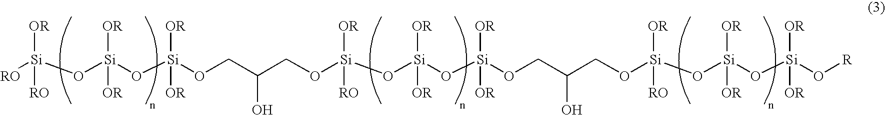

wherein R is hydrogen, —CH₂CH₃ or —CH₂CHOHCH₂OH.

Optionally the polyol-doped aerogel material is deposited on a glass substrate. A substrate may be in a form such as a filter, slide, particle, fiber, sheet, or tubing. A preferred substrate form is a bead.

Further provided is a method for detecting a biological particle in an environment, the method having several steps, including providing an inventive biological particle capture medium, placing the medium in the environment for a period of time sufficient to expose the medium to a volume of air in the environment, performing a test to detect the presence of the biological particle on the medium; and correlating the presence of the biological particle on the medium with the presence of the biological particle in the environment in order to detect the biological particle in the environment. Optionally the test to detect the biological particle includes detecting a nucleic acid sequence of the particle, particle culture and/or antigen detection.

### BRIEF DESCRIPTION OF THE DRAWINGS

FIG. 1 is a graphic representation of the efficiency of various collection systems;

FIG. 2A is a graphic representation of the viability of biological particles in various collection systems;

FIG. 2B is a graphic representation of the viability of biological particles in various collection systems;

FIG. 3A is a graphic representation of the efficiency of various collection systems;

FIG. 3B is a graphic representation of the efficiency of various collection systems; and

FIG. 4 is a graphic representation of the efficiency of various collection systems.

### DETAILED DESCRIPTION OF THE INVENTION

This invention improves upon the prior art by combining osmoprotectants and polyols with silicate materials to form media for efficient collection of biological particles. The inventive media further provide improved preservation of particle viability and improved compatibility with assays to detect particle presence and identity as described in detail below.

Glycerol is a commonly used osmoprotectant for the storage and collection of bacterial cultures. Standard microbiological protocols incorporate the use of glycerol to protect cells during freezing and storage. In one embodiment of the present invention improves upon the existing art by combining the osmoprotectant and biocompatible quality of glycerol with a silica backbone for immobilization onto collection surfaces.

The term “biological particle” as used herein is intended to mean an organism, parasite, or reproductive body entity usually visible only with a microscope, that is such as, for example, bacteria, bacterial spores, fungi, fungal spores, mycoplasma, viral particles and plant particles such as pollen grains.

The term “dopant” as used herein is intended to mean a first material which is added to a second material resulting in a change in properties of the second material. For example, a dopant may be a polyol such as glycerol, in contact with a second material, such as a silicate, to produce a material with improved biological trapping properties. Such contact may include covalent bonding of the first material to the second material. In one example, covalent bonding of a polyol to a silicate material to form an aerogel with improved biological particle trapping properties is described.

The terms “glygel” and “verigel” are intended to mean a glycerol-doped aerogel as depicted below in (3).

Preparation of Polyol-Doped Aerogel Material

A polyol-doped aerogel material has the general structure shown in (1):

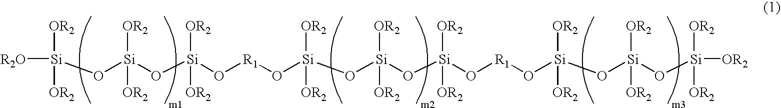

wherein the identity of R₁ depends on the polyol or polyols used in preparing the material, as well as on the identity of reagents used in the hydroysis and condensation reactions for forming the aerogel.

R₁ can be a C₁₋₂₀ alkyl, aryl, arylalkyl, substituted aryl, alkenyl, substituted alkenyl, alkynyl, substituted alkynyl, cycloalkyl or heterocycle group; optionally having one or more pendant groups which can be, for example, hydroxyl groups, C₁-C₆ linear alkyl groups such as methyl or ethyl groups; alkoxy groups, C₃₋₆ branched alkyl groups, cyclopentyl, and cyclohexyl groups.

R₂ can be hydrogen, a C₁₋₂₀ alkyl, aryl, arylalkyl, substituted aryl, alkenyl, substituted alkenyl, alkynyl, substituted alkynyl, cycloalkyl or heterocycle group; optionally having one or more pendant groups which can be, for example, hydroxyl groups, C₁-C₆ linear alkyl groups such as methyl or ethyl groups; alkoxy groups, C₃₋₆ branched alkyl groups, cyclopentyl, and cyclohexyl groups.

Preparation of a polyol-doped aerogel material includes a step of condensation of silicate backbone precursor, usually a silicon alkoxide, such as TEOS or TMOS and the like, into linear polysilicate chains, preferably in presence of a catalyst. In a further step, a polyol is added to bridge the polysilicate chains.

Thus, an inventive biological capture medium includes a polyol having the formula:

HO—R₁—OH (2)

Useful polyols of (2) include but are not limited to diols such as 1,2-ethanediol, 1,2-propanediol, 1,3-propanediol, 1,3-butanediol, 1,4-butanediol, 2,3-butanediol, 1,3-pentanediol, 1,5-pentanediol, 1,6-hexanediol, 1,3-dihydroxy-3-methylbutane, ethylene glycol, 1,10-decanediol, o-, m-, p-dihydroxycyclohexane, diethylene glycol, dipropylene glycol, 1,4-butanediol, 1,6-hexanediol and bis(2-hydroxyethyl)hydroquinone, diethylene glycol, propylene glycol, polypropylene glycol, and 2,2,4-trimethyl pentane-1,3-diol; triols such as 1,2,4-,1,3,5-trihydroxycyclohexane, glycerol and trimethylolpropane; C5-C8 sugar alcohols including sorbitan, erthyritol, pentaerthyritol, mannitol, sorbitol, xylitol, arabitol, glucose, mannose, arabinose, ribose, xylose, threose, erythrose, fructose, allose, galactose, sucrose, maltose, lactose, trehalose; and mixtures thereof.

Further, preferred polyols may include one or more heteroatom substituents in the carbon backbone, such as O, S or another bivalent heteroatom radical; a secondary or tertiary amine group.

Preferably, the polyol has an average molecular weight ranging from 60 to 1000 grams per mole, and more preferably 70 to 500 grams per mole.

A condensation step produces a product having independent chain lengths m1, m2 and m3. Chain length suitable for producing a polyol-doped aerogel product according to the present invention is preferably between 2 to 1,000-mers, more preferably in the range of 2 to 10, and even more preferably as trimers, tetramers and pentamers.

Preparation of a Glycerol-Doped Aerogel Material

Preparation of a glycerol-doped aerogel material is includes a first step of condensation of tetraethyl orthosilicate (TEOS) into short linear chains by acid catalysis as shown below.

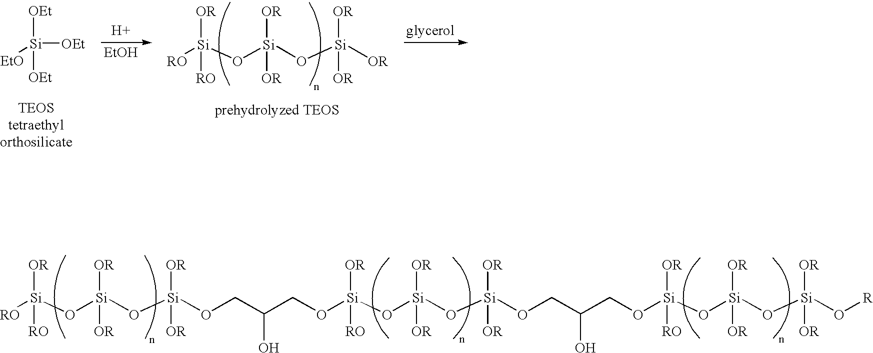

The condensation step includes mixing TEOS and an alcohol with water and an acid. The ratio of TEOS:alcohol ranges from 2:1 to 1:100, preferably from 1.9:1 to 1:10 and yet more preferably from 1.5:1 to 1:1.5. A particularly preferred ratio is 1:1.

An alcohol includes any alcohol capable of participating in the condensation reaction. A preferred alcohol is ethanol, preferably ethanol that is >90% in concentration.

An acid added to the condensation reaction mixture is preferably a strong acid such as hydrochloric acid. Preferred concentrations of the acid in the TEOS/alcohol/water mix range from 0.01 N to 0.0001 N. Preferably, the acid is present in a range from 0.005 N to 0.0005 N. A particularly preferred concentration ranges from 0.0025 N to 0.001 N, preferably about 0.0016 M HCl.

A condensation step is shown depicting a “prehydrolyzed TEOS” product having a chain length “n.” Chain length suitable for producing a glygel product according to the present invention is preferably between 2 to 1,000-mers, more preferably in the range of 2 to 10, and even more preferably as trimers, tetramers and pentamers. As indicated for the general structure (1), chain length at each site “n” is independently determined and depends on the degree of heterogeneity of oligomer length in the mix of “prehydrolyzed TEOS” or other “prehydroyzed” silicate used.

In a further step of glycerol-doped aerogel preparation, glycerol is covalently linked into the silicate backbone as shown above. The prehydrolyzed TEOS is first condensed to about ⅓-⅙ of the volume prepared as above. The solution is then heated to a temperature ranging from about 37° C. to 60° C., preferably about 50° C. Glycerol is added dropwise over a few hours resulting in a reaction mixture. The ratio of condensed prehydolyzed TEOS to glycerol is typically in the range of 1:1.2-1:1.6, preferably in the range of 1:1.3-1:1.4. The reaction mixture is then stirred at a temperature ranging from about 37° C. to 60° C., preferably about 50° C. for 40-48 hours. The reaction mixture is then condensed to approximately 90% of the prehydrolyed TEOS+glycerol total volume by rotary evaporation at about 37° C. to 60° C., preferably 50° C., and 40 torr, resulting in a condensed reaction mixture.

The condensed reaction mixture it is diluted with an equal volume of a solvent, preferably ethanol and sonicated for 20 min resulting in a stock glycerol-doped aerogel, or glygel, preparation. The stock glygel can be stored at −20° C. for months with no noticeable defects. The final solution, suitable for coating a desired substrate is prepared by diluting the stock glygel with a diluent, preferably ethanol. The degree of dilution of the stock glygel will depend on the application. It has been found that dilution of stock glygel from 1:2-1:4 with diluent results in a solution suitable for coating glass substrates, such as slides and beads.

Although an inventive biological capture medium is described above using glycerol as a dopant in a silicate backbone, the use of other polyol-type modifications of a silicate structure would be expected to offer similar benefits.

An inventive biological capture medium including the polyol dopant shown at (1) has the formula:

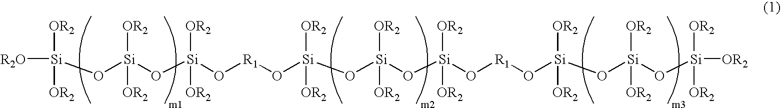

In addition, although tetraethyl orthosilicate (TEOS) is described as a useful silicate backbone for synthesis of an inventive collection surface, it is appreciated that other silicates may be used. In particular, organic silicates, such as alkyl silicates, including TMOS, TPOS and the like may be used.

In a further embodiment, a polyol or other osmoprotectant may be added to a previously synthesized aerogel to further improve biological trapping, viability or assay characteristics. For example, a glygel may be coated on a surface and glycerol, or another polyol or other osmoprotectant or mixture thereof, sprayed or otherwise dispensed onto the glygel coated surface.

Preparation of Collection Surface

A collection surface is prepared which allows a quantity of a medium putatively containing biological particles to come into contact with the surface. Typically, a collection surface is created by coating a substrate with a glygel solution. The term “coating” is intended to mean deposition of a glygel preparation onto the substrate such that the substrate is wholly or partly covered by the glygel. Coating of a substrate with a glygel is achieved by various means including dipping, spraying, painting, immersing, flow coating, and art recognized equivalents.

A glygel may be non-uniformly deposited on a substrate. A substrate may be partially coated with an inventive material for various purposes, illustratively including, to allow for exposure of the capture material to differing environments, to allow for a substrate to be fitted into a frame, and the like. A patterned deposition may be achieved by a method illustratively including ink jet printing, pin printing, and art recognized equivalents.

A substrate onto which a glygel is deposited may be any of various forms such as slides, particles such as beads, filters, fibers, sheets, and tubing. In particluar, polyol-doped aerogel coated-beads in a packed bed is an efficacious collection surface format.

A substrate may be composed of any of various materials including glass, quartz, silica, epoxies, plastics, other polymers such as nylon, cellulose, and the like. The form and composition of the substrate depend on the application. For example, where sampling of room air is desired in order to assay for presence biological particles, an air filter may be used as a substrate.

When applied to conventional filter materials or air-collection matrix, the coating increases the trapping efficiency of the filter or matrix without adversely affecting other matrix properties. For example, assuming that a pressure drop is a necessary property, this must not be adversely affected by the coating in order to maintain adequate flow through the filter. The coating described herein may be used both to trap biological particles during air collection, and to provide a viability enhancer for bacteria during air collection. The coating is also biocompatible for collection of air-borne biological particles and subsequent extraction and post-collection analysis protocols, and does not interfere with molecular analyses as described in detail below.

Curing a Collection Surface

Optionally, a collection surface is cured before exposure to media putatively containing biological particles. Curing is typically performed by exposing a coated substrate to dry for a period of time. Drying may be accomplished by known methods, illustratively including exposure to air or heat. For example, a coated substrate is air dried for a time ranging from 20 minutes to several hours followed by exposure to heat. Heat exposure may be at temperatures ranging from about 80° C. to 200° C. for a time ranging from about 15 minutes to 45 minutes. A collection surface is typically slightly tacky and hydrophilic following curing.

Biological Particle Capture Efficiency

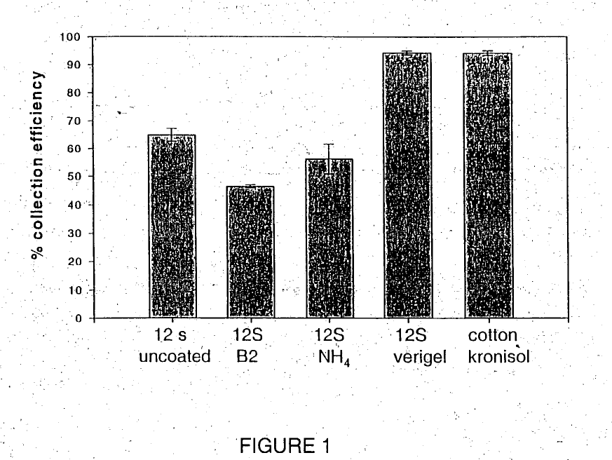

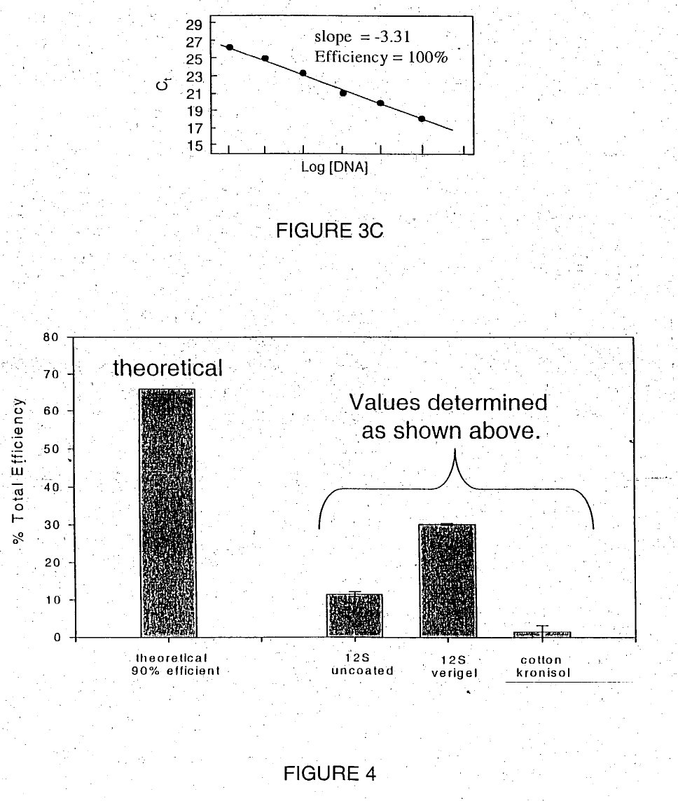

The use of glycerol as the doping material is responsible for a dramatic increase in capture efficiency and viability improvement. That is, the doping of glycerol into a silica gel aerogel coating improves trapping and keeping biological particles alive for a longer period of time. FIG. 1 illustrates results of an assay using a glycerol-doped aerogel-coated substrate as well as other types of substrate coatings and collection surfaces. The assay was performed by spraying a dose of a titered preparation of aerosolized biological particles toward one of the following: an untreated 12S filter, a B2 coated 12S filter, an ammonia coated 12S filter, a glycerol-doped aerogel (Verigel) coated filter or a kronisol coated cotton filter. In the example shown, the biological particles used were Bacillus globigii spores. A particle counter is placed in relation to the filter so that particles not retained by the filter are counted. The number of particles passing through the filter is subtracted from the total number of particles in the dose of the titered preparation. This difference is divided by the total number of particles in the dose of the titered preparation and multiplied by 100 to calculate percent collection efficiency (CE) as shown along with standard deviation (SD) in Table 1 and FIG. 1. The percent collection efficiency is also referred to as collection efficiency in FIG. 4.

TABLE 1

| filter/coating | CE | SD |
|---|---|---|
| 12S/none | 64.9 | 2.3 |
| 12S/B2 | 46.4 | 0.5 |
| 12S/NH4 | 56.2 | 5.5 |
| 12S/verigel | 94.3 | 0.7 |
| cotton/kronisol | 94.2 | 0.85 |

Biological particles captured on an inventive coating may be assessed by various assays to determine, for instance, particle number, viability and identity.

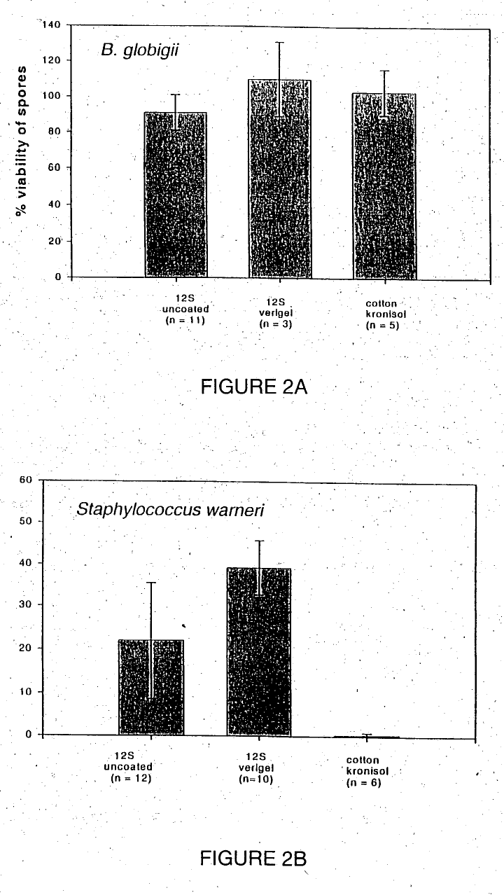

FIGS. 2A and 2B illustrate the results of one type of assay of viability of particles collected on various treated and untreated collection surfaces. The assay is performed by applying a titered biological particle preparation to an untreated 12S filter, a glycerol-doped aerogel (Verigel) coated filter or a kronisol coated cotton filter. The filter is allowed to dry for about 24 hours before the filter is broken apart, for example by using a BeadBeater (Biospec Products, Inc., Bartlesville, Okla.) for about 30 seconds. Biological particles are then separated from other particles, for example by filtration. Biological particles are then cultured by placing them in an appropriate medium and incubating for an appropriate time. In the examples shown in FIGS. 2A and 2B, titered preparations of Bacillus globigii spores (2A) or Staphylococcus warnerii (2B) were applied to filters as described above. Following separation of biological particles from filter and coating particles by filtration using a 0.2 um filter, particles were cultured on T-soy media plates. Colony forming units were counted and percent viability was calculated by determining the number of biological particles retained by the collection surface as described above and dividing the number of colony forming units by the number of biological particles retained. The percent viability is also referred to as viability efficiency in FIG. 4.

Identity of biological particles is determined by analysis of various traits such as growth medium requirements, antibiotic effectiveness and molecular characteristics. The inventive coatings facilitate particle analysis where such analysis requires separation of particles and/or particle analytes, such as nucleic acids and proteins, from the coating, since these are easily separated from the coating material. Further, the coatings do not interfere with analysis when only a partial purification of particles or analytes are performed and residual coating material remains in the assayed sample.

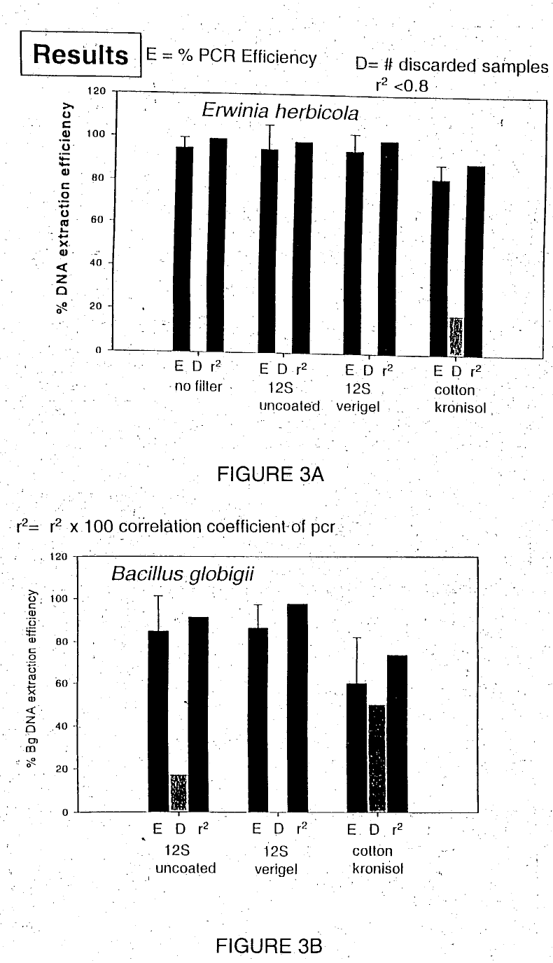

FIGS. 3A, 3B and 3C show results of real-time PCR analysis performed on DNA extracted from Erwinia herbicola cells or Bacillus globigii spores deposited on an untreated 12S filter, a glycerol-doped aerogel (Verigel) coated filter or a kronisol coated cotton filter as compared to DNA extracted from cells or spores that were not placed on a filter. Filters are dried for about 24 hours after deposition of cells or spores. In order to analyze DNA from captured biological particles, a DNA extraction method may be used to collect DNA from the particles on the capture surface. Alternatively, the particles may be separated from the collection surface by breaking up the filter and coating using a bead beat and filtration step as above, or a similar method. The captured particles are then subjected to DNA extraction and PCR analysis to determine the efficiency of DNA extraction from the collection surface and efficiency of the PCR reaction in the samples. Glycerol is also easily removed prior to analysis by well-known methods.

DNA extraction methods and PCR protocols are known in the art as are other methods for analysing molecular traits of biological particles. For instance, nucleic acids, proteins, antibiotic resistance, Gram stain pattern and the like may be examined to characterize a biological particle. Most commonly, nucleic acids are analyzed by PCR, although other techniques such as Northern and Southern blots may be used. Particle proteins may be characterized by their interaction with specific antibodies. In addition, particles may be characterized by assay for the presence of particular lectin-reactive carbohydrates or patterns of glycosylation. Exemplary techniques and protocols are found in standard references, such as, M. J. McPherson et al., PCR Basics: From Background to Bench, Springer Verlag; 2000; Chen, B.-Y. and Janes, H. W. (Eds.), PCR Cloning Protocols: From Molecular Cloning to Genetic Engineering, Humana Press, 2002; J. Sambrook et al., Molecular Cloning: A Laboratory Manual, Cold Spring Harbor Laboratory Press, 2001; E. Harlow and D. Lane (Eds.),. Antibodies: A Laboratory Manual, Cold Spring Harbor Laboratory, 1988.

FIG. 4 illustrates some advantages of using a glycerol-doped aerogel collection surface in a graphical representation of a calculation allowing comparison of efficiency of an inventive coating with other collection surfaces in promoting collection, viability and particle analysis. Overall efficiency is calculated as follows:

Eff_{F}=Eff_{C}×Eff_{V}×Eff_{DNA:C}×Eff_{DNA:S}

where Eff_{F} is the efficiency of the filter, that is, the particular combination of filter and coating, Eff_{C} is the efficiency of particle collection, Eff_{V} is the efficiency of viability, Eff_{DNA:C} is the efficiency of DNA extraction, and Eff_{DNA:S} is the efficiency of the PCR reaction in the samples. A theoretical overall efficiency is calculated for comparison. Table 2 shows values used in the calculations presented graphically in FIG. 4.

TABLE 2

| filter/coating | Eff_{C} | Eff_{V} | Eff_{DNA:C} | Eff_{DNA:S} | Eff_{F} | SD |
|---|---|---|---|---|---|---|
| 12Sfilter/ | 64.9% | 22.06% | 94.43% | 84.9% | 11.5% | ±0.65 |
| uncoated | | | | | | |
| 12Sfilter/ | 94.3% | 39.22% | 94.18% | 86.5% | 30.1% | ±0.22 |
| glygel | | | | | | |
| Cotton filter/ | 94.2% | 0.33% | 81.9% | 60% | 0.15% | ±1.62 |
| Kronisol | | | | | | |
| Theoretical | 90% | 90% | 90% | 90% | 66% | |

Comparison of an inventive glycerol-doped aerogel coating with uncoated substrates and with other coatings demonstrates the advantages of an inventive material.

A polyol-doped aerogel collection surface may be configured to collect some biological particles in preference to others. For example, where a user is interested in obtaining a environmental bacterial sample relatively free of mold, a fungicide may be included on the collection surface. Similarly, other inhibitors of certain biological particles may be used where desirable for specific applications.

Although the inventive polyol-doped aerogels are described herein as substrate coatings, it is appreciated that the polyol-doped aerogels may be used to form collection surfaces in the absence of a substrate. For example, a polyol-doped aerogel may be shaped to form slides, filters, particles such as beads, fibers, sheets, tubing and other shapes useful in the collection and analysis of biological particles. The shaped polyol-doped aerogel may be supported by a frame or holder as desired.

Method of Detecting a Biological Particle

A method for detecting a biological particle has several steps, including providing an inventive biological particle capture medium. Another step in a method includes introducing the medium into the environment or sample where a biological particle is to be detected if present. For example, the medium may be placed in a room, an air vent, a ventilation system and the like. The medium is placed in the environment to be tested for a period of time sufficient to expose the medium to a volume of air in the environment. The period of time will range from seconds to days and depends on factors such as the size of the environment and the suspected likelihood of particle presence. For example, to test a sterile hood, the medium may be placed in the hood for one or several days before analysis of particle presence since even a large volume of air in this environment is unlikely to contain biological particles.

A further step in an inventive method includes performing a test to detect the presence of the biological particle on the medium. Such tests are known in the art and include assays for detecting a nucleic acid of a particle, particle culture characteristics and detecting a particle antigen.

In an additional step of an inventive method, the presence of the biological particle on the medium may be correlated with the presence of the biological particle in the environment in order to detect the biological particle in the environment. For example, parallel tests may be run on medium placed in a control environment known to be uncontaminated by a particular biological particle type.

Similarly, a sample of air collected from an environment may be placed in contact with an inventive capture medium in order to assay biological particle presence, including quantity and type.

### EXAMPLES

### Example 1

In order to make a glycerol-doped aerogel, 2800 mL TEOS and 2800 mL of ethanol are mixed with 225 mL water and 9.2 mL 1.0 M HCl. The mixture is then slowly warmed from R.T. to 60° C. over 40 min and then maintained at 60° C. for 90 min. At the end of the 90 min, the reaction mixture is quickly cooled to RT with a chilled water recirculator. This results in prehydrolyzed TEOS. For consistency and reproducibility, temperature controlled components are used.

In a second step of production, glycerol is covalently linked into the silicate backbone. 750 mL of prehydrolyzed TEOS is condensed to 190 mL of condensed solution. The solution is then heated to 50° C. and 137.5 mL of glycerol is added dropwise over a few hours. The reaction mixture is then stirred at 50° C. for 40-48 hours. The reaction mixture is then condensed to approximately 300 mL by rotary evaporation at 50° C. and 40 torr.

The solution is diluted with 300 mL EtOH and sonicated for 20 min (stock glygel) in order to make it amenable for coating a substrate. The final solution is prepared by diluting the stock glygel 1:4 with ethanol. The stock glygel can be stored at −20° C. for months with no noticeable defects.

The above reaction can be scaled up or down and give consistent results.

Further protocols and methods for synthesis of various polyol-doped silicates are included in Gill and Ballesteros, J. Am. Chem. Soc., 1998, 120:8597-8598; C. J. Brinker, G. W. Scherer, Sol-Gel Science, Academic Press, 1990; L. C. Klein, Sol-Gel Technology for Thin Films, Fibers, Preforms, and Electronics, Noyes, 1988.

### Example 2

Experiments are performed to determine the benefits of the glygel coating in passive air collection. Glass slides are coated on both sides with a “verigel” coating and left outside (in Charlottesville, Va. during the summer). The slides are then brought inside and any culturable organisms that “stick” to the slides are washed off and cultured on bacterial culture medium. The number, diversity and type of colonies obtained are compared between a silicone slide capture surface (glass slide coated with silicone grease which is excellent for collection but exhibits a high interference in analysis), and uncoated slide capture surface. Each slide is placed in 25 mL LB medium for 15 minutes to elute captured particles. The medium is filtered and plated on nutrient agar. Colony forming units are counted and characterized visually.

### Example 3

A static decay meter was used to measure static decay characteristics of a glycerol-doped aerogel collection surface as compared to other collection surfaces. All measurements were performed by charge to +5 KV decay time measured to 10% cutoff. Three trials were performed for each surface type. The results are presented in Table 3 below.

TABLE 3

| surface type | +5 KV | −5 KV |
|---|---|---|
| 12S filter/B2 | +400 | 183, 184, 183 |
| 10% ammonia | 2.44, 2.54, 2.58 | 2.75, 2.74, 2.79 |
| 20% ammonia | .84, .84, .85 | .87, .86, .86 |
| glygel pvp | .02, .02, .01 | .02, .02, .01 |
| masterflow 100 | 13.38, 12.66, 12.86 | 12.37, 11.97, 12.27 |
| electrostat 90 | ND | |
| PLEDGE GRAB IT | 9.77, 9.85, 10.20 | 10.15, 10.11, 10.31 |
| 12S filter/wet | .01, .01, .01 | .01, .01, .01 |

| | | |
|---|---|---|
| Cotton | 7.62, 7.52, 7.53 | 7.10, 7.01, 6.97 |
| cotton/kronisol | 24.66, 24.6, 25.88 | 20.62, 21.01, 20.47 |
| dry glygel | .05, .05, .04 | .02, .02, .02 |
| Glygel | .04, .04, .04 | .05, .05, .05 |
| B2-PVP 1% | +400 | 85.41, 84.71, 88.17 |
| Remay 2040 | +400 (3 V) | (2 V) won't take |
| Remay 2040-glygel | 8.4, 8.2, 8.23 | 6.67, 6.54, 6.02 |

### Example 4

Coating of glass beads is accomplished using 107 g of washed 500 um glass beads. Beads are split into 3 portions and washed overnight in glygel. Beads are then filtered to remove the sol and cured at 100° C. for 30 minutes.

### Example 5

Glass filter strips are coated with glygel by dipping followed by air drying for 60 minutes. The strips are then cured at 100° C. for 30 minutes.

### Example 6

Filters are coated with glygel by dipping followed by air drying for 60 minutes. The strips are then cured at 85° C. for 18 minutes.

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

One skilled in the art will readily appreciate that the present invention is well adapted to carry out the objects and obtain the ends and advantages mentioned, as well as those inherent therein. The present methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

We claim:

1. An improved biological particle capture medium, comprising:
a polyol-doped aerogel deposited on a substrate.

2. The improved biological capture medium of claim 1, wherein the polyol-doped aerogel comprises an oligomeric silicate having the formula 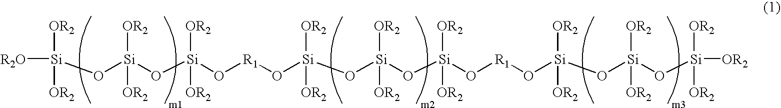
wherein R1 and R2 are each independently a C₁₋₂₀ alkyl, aryl, arylalkyl, substituted aryl, alkenyl, substituted alkenyl, alkynyl, substituted alkynyl, cycloalkyl or heterocycle group and wherein R2 may be hydrogen.

3. The improved biological capture medium of claim 2, wherein R1 or R2 further comprises a pendant moiety selected from the group consisting of: hydroxyl group, C₁-C₆ linear alkyl group, alkoxy group, C₃₋₆ branched alkyl group, cyclopentyl group, and cyclohexyl group.

4. The improved biological capture medium of claim 1, wherein the polyol-doped aerogel comprises a glyceryl-bridged oligomeric silicate having the formula 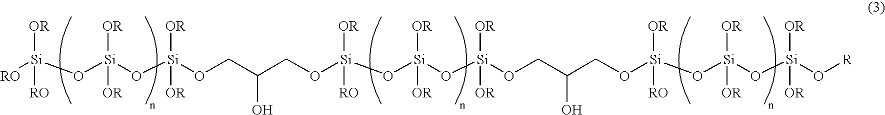
wherein R is hydrogen, —CH₂CH₃ or —CH₂CHOHCH₂OH.

5. The improved biological capture medium of claim 1, wherein the polyol-doped aerogel deposited on a glass substrate.

6. The improved biological capture medium of claim 1, wherein the substrate is a filter.

7. The improved biological capture medium of claim 1, wherein the substrate is selected from a group consisting of: slide, particle, fiber, sheet, and tubing.

8. The improved biological capture medium of claim 5, wherein the substrate particle is a bead.

9. A method for detecting a biological particle in an environment, the method comprising the steps of:
providing the biological particle capture medium of claim 1;
placing the medium in the environment for a period of time sufficient to expose the medium to a volume of air in the environment;
performing a test to detect the presence of the biological particle on the medium; and
correlating the presence of the biological particle on the medium with the presence of the biological particle in the environment in order to detect the biological particle in the environment.

10. The method for detecting a biological particle of claim 7, wherein the test comprises nucleic acid detection.

11. The method for detecting a biological particle of claim 7, wherein the test comprises particle culture.

12. The method for detecting a biological particle of claim 7, wherein the test comprises antigen detection.

13. A method for detecting a biological particle in a sample, the method comprising the steps of:
providing the biological particle capture medium of claim 1;
placing the medium in contact with the sample;
performing a test to detect a captured biological particle on the medium; and correlating detection of the captured biological particle on the medium with presence of the biological particle in the sample.

14. An improved biological particle capture surface, comprising:
a silica gel aerogel coating; and
a glycerol dopant.

15. The improved biological capture surface of claim 14, wherein the coating is disposed on a glass substrate.

16. The improved biological capture surface of claim 14, wherein the coating is applied to beads.

17. A method for bioassay, comprising the steps of:
providing a filter comprising an aerogel and glycerol;
placing the filter in an environment to be assayed for a period of time sufficient to expose the filter to a volume of air in the environment;
removing the filter from the environment; and
performing a test to determine the presence of a biological particle on the filter.

18. The method for bioassay of claim 17, wherein the test comprises nucleic acid detection.

19. The method for bioassay of claim 17, wherein the test comprises particle culture.
